(19) United States
(12) Patent Application Publication
(10) Pub. No.: US 2026/0315034 A1
(43) Pub. Date: Oct. 15, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Tsuyoshi MATSUMOTO, Kanagawa (JP)

(73) Assignee: FUJIFILM Corporation, Tokyo (JP)

(21) Appl. No.: 19/570,168

(22) Filed: Mar. 18, 2026

(30) Foreign Application Priority Data
Apr. 10, 2025 (JP) 2025-064980

(51) Int. Cl. A61B 8/00; A61B 8/08

(52) U.S. Cl. CPC A61B 8/5207; A61B 8/0825; A61B 8/463; A61B 8/54

(57) ABSTRACT

Provided are an ultrasound diagnostic apparatus and a control method of an ultrasound diagnostic apparatus that can reliably examine a tomographic plane observed in a past ultrasound examination.

An ultrasound diagnostic apparatus includes an image acquisition unit that acquires an ultrasound image by transmitting and receiving an ultrasound beam from an ultrasound probe, a probe position information acquisition unit that acquires position information of the ultrasound probe, a path information generation unit that generates path information of the ultrasound probe based on the position information, a scanning path coincidence determination unit that determines, based on the path information, whether or not a current scanning path coincides with a past scanning path, an image coincidence determination unit that, in a case where it is determined that the current scanning path coincides with the past scanning path, determines whether or not a current ultrasound image coincides with a past ultrasound image, and a notification unit that notifies a user of a determination result.

### CROSS-REFERENCE TO RELATED APPLICATIONS

The present application claims priority under 35 U.S.C. § 119 to Japanese Patent Application No. 2025-064980, filed on Apr. 10, 2025. Each of the above application(s) is hereby expressly incorporated by reference, in its entirety, into the present application.

### BACKGROUND OF THE INVENTION

### 1. Field of the Invention

The present invention relates to an ultrasound diagnostic apparatus and a control method of an ultrasound diagnostic apparatus for performing an ultrasound examination a breast of a subject.

### 2. Description of the Related Art

In the related art, an ultrasound examination has been performed in which an ultrasound image representing a tomographic plane of a subject is captured using a so-called ultrasound diagnostic apparatus, and a lesion or the like in the subject is examined based on the captured ultrasound image. For a subject in which a portion representing a lesion, a portion suspected to be a lesion, or a portion to be carefully examined is found in a past examination, for example, a continuous ultrasound examination of the portion to be carefully examined may be performed. In a case where there are a plurality of portions to be carefully examined, it may be difficult for a user such as a doctor to perform an ultrasound examination on all the portions to be carefully examined. Therefore, for example, a technique disclosed in JP2014-036754A has been developed to enable an ultrasound examination under the same examination conditions as those in a past examination. In JP2014-036754A, an examination is performed in a case where a relative position of an ultrasound probe with respect to a subject coincides a relative position in a past examination.

### SUMMARY OF THE INVENTION

Meanwhile, an ultrasound examination may be performed on a breast of a subject. The breast is usually easily deformed due to a body position or a body movement of the subject. Therefore, even in a case where it is possible to confirm that the relative position of the ultrasound probe with respect to the subject coincides with the relative position in the past examination using the technique of JP2014-036754A, the tomographic plane captured in the past examination and the tomographic plane currently being captured may be different from each other due to the deformation of the breast. In this case, the user may erroneously recognize that the tomographic plane captured in the past examination is observed, and may end the ultrasound examination of the breast without examining the portion to be carefully examined in the breast.

The present invention has been made in order to solve such a problem in the related art, and an object of the present invention is to provide an ultrasound diagnostic apparatus and a control method of an ultrasound diagnostic apparatus that can reliably examine a tomographic plane observed in a past ultrasound examination.

The above-described object can be achieved with the following configurations.
[1] An ultrasound diagnostic apparatus that performs an ultrasound examination of a breast of a subject by scanning the breast with an ultrasound probe, the ultrasound diagnostic apparatus comprising:
an image acquisition unit that acquires an ultrasound image by transmitting and receiving an ultrasound beam using the ultrasound probe;
a probe position information acquisition unit that acquires position information of the ultrasound probe;
a scanning method specifying unit that specifies a scanning method of the ultrasound probe with respect to the breast;
a path information generation unit that generates, based on the position information acquired by the probe position information acquisition unit, path information related to a scanning path of the ultrasound probe used for scanning in accordance with the scanning method specified by the scanning method specifying unit;
a scanning path coincidence determination unit that determines, based on the path information generated by the path information generation unit, whether or not a current scanning path coincides with a past scanning path in a past ultrasound examination of the subject;
an image coincidence determination unit that, in a case where it is determined by the scanning path coincidence determination unit that the current scanning path coincides with the past scanning path, determines whether or not a current ultrasound image acquired by the image acquisition unit coincides with a past ultrasound image acquired by the image acquisition unit in the past ultrasound examination of the subject; and
a notification unit that notifies a user of a determination result by the image coincidence determination unit.
[2] The ultrasound diagnostic apparatus according to [1], further comprising: an image memory in which the ultrasound image acquired by the image acquisition unit is stored; and
   a path information memory in which the path information generated by the path information generation unit is stored,
in which the scanning path coincidence determination unit determines whether or not the current scanning path coincides with the past scanning path based on the path information stored in the path information memory, and
the image coincidence determination unit determines whether or not the current ultrasound image coincides with the past ultrasound image based on the ultrasound image stored in the image memory.
[3] The ultrasound diagnostic apparatus according to [1] or [2], further comprising:
an input device for a user to perform an input operation,
in which the scanning method specifying unit specifies the scanning method based on information input by the user via the input device.
[4] The ultrasound diagnostic apparatus according to [1] or [2],
in which the scanning method specifying unit specifies the scanning method based on at least one of the ultrasound image acquired by the image acquisition unit or the position information acquired by the probe position information acquisition unit.
[5] The ultrasound diagnostic apparatus according to any one of [1] to [4],
in which the path information generation unit generates the path information based on the position information continuously acquired by the probe position information acquisition unit in association with scanning using the ultrasound probe.
[6] The ultrasound diagnostic apparatus according to any one of [1] to [4], in which the path information generation unit generates the path information based on discrete position information acquired by the probe position information acquisition unit.
[7] The ultrasound diagnostic apparatus according to any one of [1] to [6],
in which the image coincidence determination unit compares the current ultrasound image acquired by the image acquisition unit with the past ultrasound image acquired by the image acquisition unit, at at least one location corresponding to the scanning path determined to coincide by the scanning path coincidence determination unit, to determine coincidence between the current ultrasound image and the past ultrasound image.
[9] The ultrasound diagnostic apparatus according to any one of [1] to [6], further comprising:
a deformation detection unit that detects deformation of the breast of the subject; and a path information correction unit that corrects current path information generated by the path information generation unit based on the deformation of the breast detected by the deformation detection unit,
in which the scanning path coincidence determination unit determines coincidence of the scanning path based on the current path information corrected by the path information correction unit and past path information.
[9] The ultrasound diagnostic apparatus according to any one of [1] to [8], further comprising:
a monitor,
in which the notification unit displays, side by side on the monitor, the current ultrasound image acquired by the image acquisition unit and the past ultrasound image acquired by the image acquisition unit and highlights portions that differ from each other.
[10] The ultrasound diagnostic apparatus according to any one of [1] to [9], further comprising:
a path information correction unit that, in a case where the current scanning path deviates from the past scanning path by a predetermined threshold value or more, corrects the path information generated by the path information generation unit based on the past scanning path and proposes the corrected path information to the user.
[11] The ultrasound diagnostic apparatus according to any one of [1] to [10],
in which the ultrasound image is any of a B-mode image, a Doppler image, or an elasticity image.
[12] A control method for an ultrasound diagnostic apparatus that performs an ultrasound examination of a breast of a subject by scanning the breast with an ultrasound probe, the control method comprising:
acquiring an ultrasound image by transmitting and receiving an ultrasound beam using the ultrasound probe;
acquiring position information of the ultrasound probe;
specifying a scanning method of the ultrasound probe with respect to the breast;
generating, based on the acquired position information, path information related to a scanning path of the ultrasound probe used for scanning in accordance with the specified scanning method;
determining, based on the generated path information, whether or not a current scanning path coincides with a past scanning path in a past ultrasound examination of the subject;
in a case where it is determined that the current scanning path coincides with the past scanning path, determining whether or not an acquired current ultrasound image coincides with a past ultrasound image acquired in the past ultrasound examination of the subject; and notifying a user of a determination result of coincidence between the current ultrasound image and the past ultrasound image.

According to the present invention, there is provided an ultrasound diagnostic apparatus that performs an ultrasound examination of a breast of a subject by scanning the breast with an ultrasound probe, the ultrasound diagnostic apparatus comprising an image acquisition unit that acquires an ultrasound image by transmitting and receiving an ultrasound beam using the ultrasound probe; a probe position information acquisition unit that acquires position information of the ultrasound probe; a scanning method specifying unit that specifies a scanning method of the ultrasound probe with respect to the breast; a path information generation unit that generates, based on the position information acquired by the probe position information acquisition unit, path information related to a scanning path of the ultrasound probe used for scanning in accordance with the scanning method specified by the scanning method specifying unit; a scanning path coincidence determination unit that determines, based on the path information generated by the path information generation unit, whether or not a current scanning path coincides with a past scanning path in a past ultrasound examination of the subject; an image coincidence determination unit that, in a case where it is determined by the scanning path coincidence determination unit that the current scanning path coincides with the past scanning path, determines whether or not a current ultrasound image acquired by the image acquisition unit coincides with a past ultrasound image acquired by the image acquisition unit in the past ultrasound examination of the subject; and a notification unit that notifies a user of a determination result by the image coincidence determination unit. Therefore, it is possible to reliably examine a tomographic plane observed in the past ultrasound examination.

### BRIEF DESCRIPTION OF THE DRAWINGS

FIG. 1 is a block diagram showing a configuration of an ultrasound diagnostic apparatus according to a first embodiment of the present invention.

FIG. 2 is a block diagram illustrating an internal configuration of a transmission and reception circuit in the first embodiment of the present invention.

FIG. 3 is a block diagram illustrating an internal configuration of an image generation unit according to the first embodiment of the invention.

FIG. 4 is a diagram showing an ideal scanning path of an ultrasound probe in a first example of a scanning method of a breast.

FIG. 5 is a diagram showing an ideal scanning path of the ultrasound probe in a second example of the scanning method of the breast.

FIG. 6 is a diagram showing an ideal scanning path of the ultrasound probe in a third example of the scanning method of the breast.

FIG. 7 is a diagram showing an ideal scanning path of the ultrasound probe in a fourth example of the scanning method of the breast.

FIG. 8 is a diagram showing a display example of a past ultrasound image and a current ultrasound image at the same examination position.

FIG. 9 is a flowchart showing the operation of the ultrasound diagnostic apparatus according to the first embodiment of the invention.

FIG. 10 is a block diagram showing an internal configuration of an image generation unit in a first modification example of the first embodiment of the present invention.

FIG. 11 is a block diagram showing an internal configuration of the image generation unit in a second modification example of the first embodiment of the present invention.

FIG. 12 is a block diagram showing a configuration of an ultrasound diagnostic apparatus according to a second embodiment of the present invention.

FIG. 13 is a block diagram illustrating a configuration of an ultrasound diagnostic apparatus according to a third embodiment of the present invention.

### DESCRIPTION OF THE PREFERRED EMBODIMENTS

Hereinafter, an embodiment of the present invention will be described with reference to the accompanying drawings.

Description of configuration requirements written below is provided based on a representative embodiment of the present invention, but the present invention is not limited to such an embodiment.

In the present specification, numerical ranges represented by “to” include numerical values before and after “to” as lower limit values and upper limit values.

In the present specification, “the same” includes an error range generally allowed in the technical field.

### First Embodiment

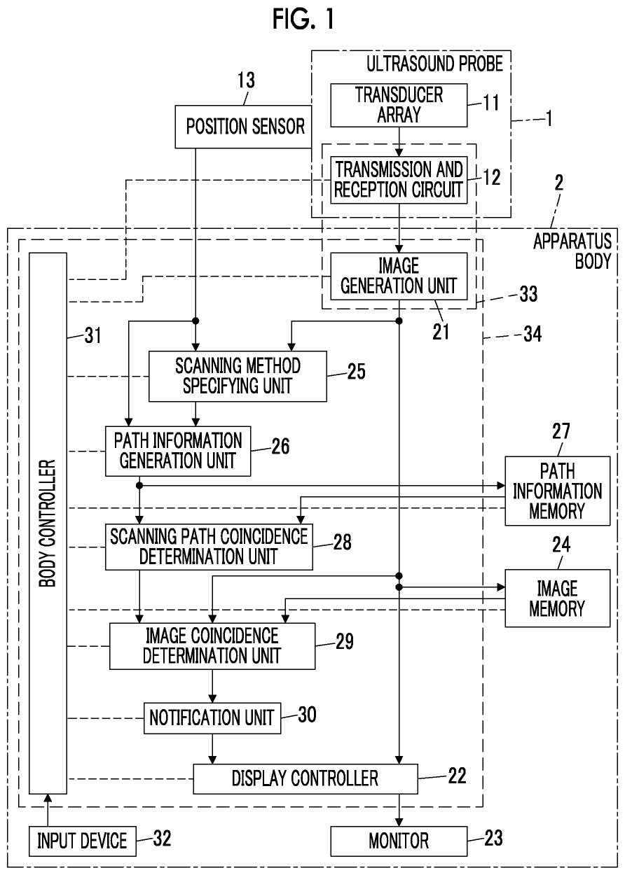

FIG. 1 shows a configuration of an ultrasound diagnostic apparatus according to a first embodiment of the present invention. The ultrasound diagnostic apparatus comprises an ultrasound probe 1 and an apparatus body 2 connected to each other by so-called wired communication or so-called wireless communication.

The ultrasound probe 1 comprises a transducer array 11 and a transmission and reception circuit 12 connected to the transducer array 11. In addition, a position sensor 13 is attached to the ultrasound probe 1.

The apparatus body 2 comprises an image generation unit 21 connected to the transmission and reception circuit 12. In the apparatus body 2, a display controller 22 and a monitor 23 are sequentially connected to the image generation unit 21. In addition, an image memory 24 is connected to the image generation unit 21. A scanning method specifying unit 25 is connected to the image generation unit 21 and the position sensor 13. A path information generation unit 26 is connected to the scanning method specifying unit 25 and the position sensor 13. A path information memory 27 is connected to the path information generation unit 26. A scanning path coincidence determination unit 28 is connected to the path information generation unit 26 and the path information memory 27. An image coincidence determination unit 29 is connected to the image generation unit 21, the image memory 24, and the scanning path coincidence determination unit 28. A notification unit 30 is connected to the image coincidence determination unit 29. The notification unit 30 is connected to the display controller 22. In addition, a body controller 31 is connected to the transmission and reception circuit 12, the image generation unit 21, the display controller 22, the image memory 24, the scanning method specifying unit 25, the path information generation unit 26, the path information memory 27, the scanning path coincidence determination unit 28, the image coincidence determination unit 29, and the notification unit 30. An input device 32 is connected to the body controller 31.

The transmission and reception circuit 12 and the image generation unit 21 constitute an image acquisition unit 33. A processor 34 for the apparatus body 2 is configured by the image generation unit 21, the display controller 22, the scanning method specifying unit 25, the path information generation unit 26, the scanning path coincidence determination unit 28, the image coincidence determination unit 29, the notification unit 30, and the body controller 31.

The transducer array 11 of the ultrasound probe 1 has a plurality of ultrasound transducers that are one-dimensionally or two-dimensionally arranged. In accordance with a drive signal supplied from the transmission and reception circuit 12, each of the ultrasound transducers transmits ultrasound and receives an ultrasound echo from a subject to output a signal based on the ultrasound echo. Each ultrasound transducer is configured by forming an electrode at both ends of a piezoelectric body consisting of, for example, a piezoelectric ceramic represented by lead zirconate titanate (PZT), a polymer piezoelectric element represented by polyvinylidene difluoride (PVDF), and a piezoelectric single crystal represented by a lead magnesium niobate-lead titanate (PMN-PT).

The image acquisition unit 33 configured by the transmission and reception circuit 12 and the image generation unit 21 acquires an ultrasound image in which a breast of a subject is imaged by transmitting and receiving an ultrasound beam using the ultrasound probe 1.

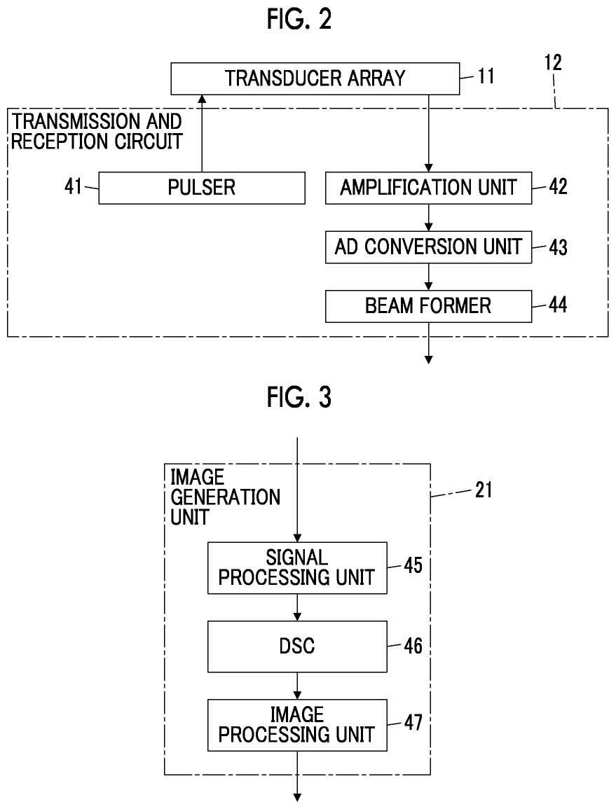

The transmission and reception circuit 12 transmits the ultrasound wave from the transducer array 11 and generates a sound ray signal based on a reception signal acquired by the transducer array 11, under the control of the body controller 31. As shown in FIG. 2, the transmission and reception circuit 12 includes a pulser 41 connected to the transducer array 11, and an amplification unit 42, an analog-to-digital (AD) conversion unit 43, and a beam former 44 that are sequentially connected in series to the transducer array 11.

The pulser 41 includes, for example, a plurality of pulse generators, and adjusts an amount of delay of each of drive signals and supplies the drive signals to the plurality of ultrasound transducers such that ultrasound waves transmitted from the plurality of ultrasound transducers of the transducer array 11 form an ultrasound beam based on a transmission delay pattern selected according to a control signal from the body controller 31. In this way, in a case where a pulsed or continuous wave-like voltage is applied to the electrodes of the ultrasound transducer of the transducer array 11, the piezoelectric body expands and contracts to generate a pulsed or continuous wave-like ultrasound wave from each of the ultrasound transducers, thereby forming an ultrasound beam from the combined wave of these ultrasound waves.

The transmitted ultrasound beam is reflected in a target, for example, a site of the subject, and propagates toward the transducer array 11 of the ultrasound probe 1. The ultrasound echo propagating toward the transducer array 11 is received by each ultrasound transducer constituting the transducer array 11. In this case, each of the ultrasound transducers constituting the transducer array 11 receives the propagating ultrasound echo to expand and contract to generate the reception signal, which is an electrical signal, and outputs these reception signals to the amplification unit 42.

The amplification unit 42 amplifies the signal input from each of the ultrasound transducers constituting the transducer array 11 and transmits the amplified signal to the AD conversion unit 43. The AD conversion unit 43 converts the signal transmitted from the amplification unit 42 into digital reception data. The beam former 44 performs so-called reception focus processing by applying and adding the delay to each reception data received from the AD conversion unit 43. By this reception focus processing, each reception data converted by the AD conversion unit 43 is phase-added, and the sound ray signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series to each other.

The signal processing unit 45 generates a B-mode image signal, which is tomographic image information regarding tissues within the subject, by performing, on the sound ray signal received from the transmission and reception circuit 12, correction of the attenuation due to the distance according to the depth of the reflection position of the ultrasound wave using a sound velocity value set by the body controller 31 and then performing envelope detection processing.

The DSC 46 converts (raster-converts) the B-mode image signal generated by the signal processing unit 45 into an image signal in accordance with a normal television signal scanning method.

The image processing unit 47 performs various types of necessary image processing such as gradation processing on the B-mode image signal input from the DSC 46, and then transmits the B-mode image signal to the display controller 22, the image memory 24, and the scanning method specifying unit 25. Hereinafter, the B-mode image signal that is image-processed by the image processing unit 47 will be referred to as an ultrasound image.

The image memory 24 is a memory that stores the ultrasound images of the plurality of frames acquired by the image acquisition unit 33. The ultrasound image of at least one frame among the ultrasound images of the plurality of frames stored in the image memory 24 is read out under the control of the body controller 31 and transmitted to the image coincidence determination unit 29. Here, as the image memory 24, for example, a recording medium such as a flash memory, a hard disk drive (HDD), a solid-state drive (SSD), a flexible disk (FD), a magneto-optical disk (MO disk), a magnetic tape (MT), a random-access memory (RAM), a compact disc (CD), a digital versatile disc (DVD), a secure digital card (SD card), or a universal serial bus memory (USB memory) can be used.

The position sensor 13 is a sensor device that acquires position information of the ultrasound probe 1. The position information of the ultrasound probe 1 detected by the position sensor 13 can include, for example, position coordinates of the ultrasound probe 1 in a three-dimensional space and a posture of the ultrasound probe 1 in the three-dimensional space. The posture of the ultrasound probe 1 in the three-dimensional space is represented by, for example, an inclination angle of the ultrasound probe 1 in the three-dimensional space. As the position sensor 13, for example, a so-called magnetic sensor, an acceleration sensor, a gyro sensor, or a global positioning system (GPS) sensor can be used, and these are attached to the housing of the ultrasound probe 1 from the outside or are built in the ultrasound probe 1, for example.

FIG. 1 shows an example in which the position sensor 13 is attached to the ultrasound probe 1, but the position sensor 13 may be installed independently of the ultrasound probe 1. In this case, as the position sensor 13, for example, a so-called optical camera, a laser distance-measuring sensor, or a time of flight (ToF) sensor can be used. The information on the position of the ultrasound probe 1 detected by the position sensor 13 is transmitted to the scanning method specifying unit 25.

The scanning method specifying unit 25 specifies a scanning method of the ultrasound probe 1 for the breast. The scanning method of the ultrasound probe 1 for the breast refers to a method of performing scanning while moving the ultrasound probe 1 along a predetermined scanning path for the breast. For example, a plurality of scanning methods of the ultrasound probe 1 and an examination protocol in which the plurality of scanning methods are combined are determined by a breast ultrasound diagnosis guideline or the like created by The Japan Association of Breast and Thyroid Sonology (JABTS) or the like.

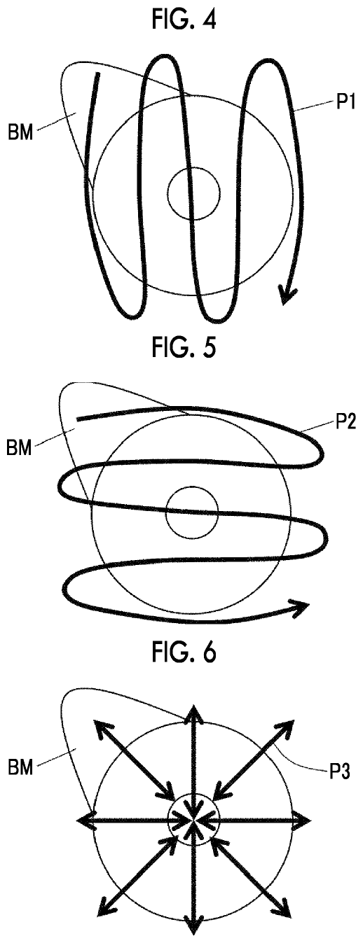

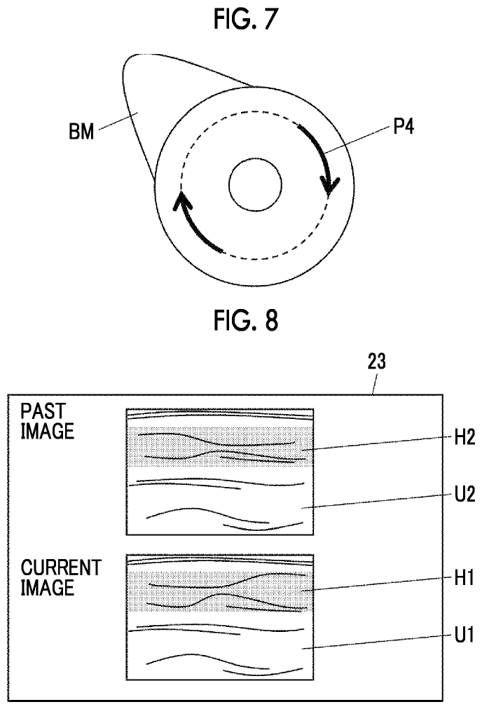

As the scanning method of the ultrasound probe 1, for example, as shown in FIG. 4, a scanning method is known in which the ultrasound probe 1 is moved along a path P1 that proceeds in a lateral direction orthogonal to a body length direction while crossing a breast of a subject a plurality of times in a meandering manner along the body length direction toward a head side and a foot side of the subject. In addition, for example, as shown in FIG. 5, a scanning method is known in which the ultrasound probe 1 is moved along a path P2 that proceeds in the body length direction of the subject while crossing the breast of the subject a plurality of times in a meandering manner along a body width direction of the subject. In addition, as shown in FIG. 6, a scanning method is known in which the ultrasound probe 1 is moved along a radial path P3 centered on a position of a nipple of the breast. In addition, for example, as shown in FIG. 7, a scanning method is known in which the ultrasound probe 1 is moved along a circular path P4 centered on the position of the nipple of the breast. In FIGS. 4 to 7, for convenience, each of the paths P1 to P4 on the breast of the subject is shown using a body mark BM representing a right breast of the subject.

The scanning method specifying unit 25 stores start positions and postures of the ultrasound probe 1 in the plurality of scanning methods in advance, and can compare the position and the posture of the ultrasound probe 1 in the three-dimensional space detected by the position sensor 13 with the start positions and the postures of the ultrasound probe 1 in the plurality of scanning methods stored in advance. In this case, the scanning method specifying unit 25 can specify the scanning method corresponding to the start position and the posture of the ultrasound probe 1 that coincide with the position and the posture of the ultrasound probe 1 in the three-dimensional space detected by the position sensor 13, as the current scanning method of the ultrasound probe 1.

The scanning method specifying unit 25 can also store the paths in the plurality of scanning methods in advance, for example. In this case, the scanning method specifying unit 25 can generate a trajectory of the ultrasound probe 1 within a predetermined time from the start of scanning based on the position of the ultrasound probe 1 detected by the position sensor 13, for example, and can compare the generated trajectory with the plurality of paths in the plurality of scanning methods stored in advance. The scanning method specifying unit 25 can calculate a similarity between the generated trajectory and the plurality of paths in the plurality of scanning methods using, for example, a so-called pattern matching method or the like, and can specify the scanning method corresponding to the path having the highest similarity, as the current scanning method of the ultrasound probe 1.

The scanning method specifying unit 25 can also store, for example, a plurality of typical reference ultrasound images corresponding to the start positions and the postures of the ultrasound probe 1 in the plurality of scanning methods in advance, and can compare the plurality of reference ultrasound images with the ultrasound image acquired at the start of scanning with each other. The scanning method specifying unit 25 can calculate a similarity between the plurality of reference ultrasound images and the ultrasound image acquired at the start of scanning using, for example, a pattern matching method or the like, and can specify the scanning method corresponding to the reference ultrasound image having the highest similarity, as the current scanning method of the ultrasound probe 1.

The scanning method specifying unit 25 can also store, for example, a plurality of typical reference ultrasound images obtained in the plurality of scanning methods and a plurality of positions of the ultrasound probe 1 for capturing the plurality of reference ultrasound images in advance. In this case, the scanning method specifying unit 25 can estimate the position of the ultrasound probe 1 corresponding to the acquired ultrasound image by comparing, for example, the plurality of reference ultrasound images with the ultrasound image acquired in the current ultrasound examination, and can specify the scanning method corresponding to the path including the estimated positions of the ultrasound probe 1 equal to or greater than a predetermined number, as the current scanning method of the ultrasound probe 1.

The scanning method specifying unit 25 can also specify the current scanning method based on, for example, information input by the user via the input device 32.

The path information generation unit 26 generates path information related to a scanning path of the ultrasound probe 1 that is used for scanning in accordance with the scanning method specified by the scanning method specifying unit 25 based on the position information acquired by the position sensor 13. The path information related to the scanning path of the ultrasound probe 1 that is used for scanning in accordance with the specified scanning method includes a plurality of consecutive position coordinates of the ultrasound probe 1 acquired by the position sensor 13, or the position coordinates of the ultrasound probe 1 acquired by the position sensor 13 corresponding to the inflection points of the predetermined path corresponding to the scanning method specified by the scanning method specifying unit 25.

The path information memory 27 is a memory that stores the path information generated by the path information generation unit 26 in the past examination and the current examination. The path information memory 27 can read out the past path information generated by the path information generation unit 26 for the scanning of the ultrasound probe 1 in the past examination according to the same scanning method as the current scanning method specified by the scanning method specifying unit 25, under the control of the body controller 31. The past path information read out in this way is sent to the scanning path coincidence determination unit 28. As the path information memory 27, for example, a recording medium such as a flash memory, an HDD, an SSD, an FD, an MO disk, an MT, a RAM, a CD, a DVD, an SD card, or a USB memory can be used.

The scanning path coincidence determination unit 28 determines whether or not the current scanning path coincides with the past scanning path in the past ultrasound examination of the subject based on the path information generated by the path information generation unit 26. The scanning path coincidence determination unit 28 can calculate, for example, a difference between the current scanning path and the past scanning path, and determine that the current scanning path coincides with the past scanning path in a case where the calculated difference is equal to or less than a predetermined difference threshold value.

In a case where the plurality of consecutive position coordinates of the ultrasound probe 1 are used as the path information, the scanning path coincidence determination unit 28 can calculate, for example, a difference value between the position coordinates corresponding to each other in the plurality of current position coordinates and the plurality of past position coordinates, and calculate a statistical value such as an average value or a median value of the plurality of calculated difference values as a difference between the current scanning path and the past scanning path. The position coordinates corresponding to each other refer to a combination of position coordinates having the closest values in the plurality of current position coordinates and the plurality of past position coordinates.

In a case where the position coordinates of the ultrasound probe 1 corresponding to the inflection points of the path of the scanning method specified as the path information are used, the scanning path coincidence determination unit 28 can calculate, for example, a difference value between the position coordinates of the current ultrasound probe 1 corresponding to the inflection points and the position coordinates of the past ultrasound probe 1 corresponding to the inflection points as a difference between the current scanning path and the past scanning path.

In addition, the scanning path coincidence determination unit 28 can generate the current scanning path from the path information generated by the path information generation unit 26, generate the past scanning path from the past path information read out from the path information memory 27, calculate a similarity between a shape of the current scanning path and a shape of the past scanning path by a pattern matching method or the like, and determine that the current scanning path coincides with the past scanning path in a case where the calculated similarity exceeds a predetermined similarity threshold value. The scanning path coincidence determination unit 28 can also read out the past scanning path generated in the past examination from the path information memory 27 instead of generating the past scanning path from the past path information.

In a case where the scanning path coincidence determination unit 28 determines that the current scanning path coincides with the past scanning path, the image coincidence determination unit 29 determines whether or not the current ultrasound image acquired by the image acquisition unit 33 coincides with the past ultrasound image acquired by the image acquisition unit 33 in the past ultrasound examination of the subject. The image coincidence determination unit 29 can calculate a similarity between the current ultrasound image and the past ultrasound image by, for example, pattern coincidence or the like, and determine that the current ultrasound image coincides with the past ultrasound image in a case where the calculated similarity exceeds a predetermined similarity threshold value.

The image coincidence determination unit 29 can extract a plurality of ultrasound images corresponding to a plurality of positions arranged at equal intervals in each of the current scanning path and the past scanning path, and determine coincidence between the plurality of extracted current ultrasound images and the plurality of extracted past ultrasound images. In a case where all of the plurality of current ultrasound images coincide with the corresponding past ultrasound images, the image coincidence determination unit 29 can make a final determination that the current ultrasound image coincides with the past ultrasound image. In a case where at least one of the plurality of current ultrasound images does not coincide with the corresponding past ultrasound image, the image coincidence determination unit 29 can make a final determination that the current ultrasound image does not match the past ultrasound image.

The image coincidence determination unit 29 can extract at least one ultrasound image corresponding to an inflection point of the scanning path in each of the current scanning path and the past scanning path, and determine coincidence between the at least one extracted current ultrasound image and the at least one extracted past ultrasound image.

In addition, the image coincidence determination unit 29 can perform processing of detecting a characteristic structure from the current ultrasound images of the plurality of frames by analyzing the current ultrasound images of the plurality of frames, and extract at least one ultrasound image from which the characteristic structure can be detected. The image coincidence determination unit 29 can further determine coincidence between the at least one extracted ultrasound image and the corresponding past ultrasound image. The characteristic structure to be detected can include a structure representing a lesion and a structure suspected to be a lesion, in addition to a characteristic anatomical structure originally present in the breast of the subject. The image coincidence determination unit 29 can detect the characteristic structure from the ultrasound image by a so-called template coincidence method, a method of inputting the ultrasound image to a trained model in so-called machine learning in which a relationship between a large number of ultrasound images and the characteristic structures shown in the ultrasound images is to be learned, or the like.

In addition, the image coincidence determination unit 29 can calculate a similarity between all of the current ultrasound images and the corresponding past ultrasound images by, for example, a pattern matching method, and extract a set of at least one current ultrasound image and a past ultrasound image in which the calculated similarity is equal to or less than a predetermined first similarity threshold value. In a case where all of the similarities of the at least one extracted set exceed a predetermined second similarity threshold value, the image coincidence determination unit 29 can make a final determination that the current ultrasound image coincides with the past ultrasound image.

The notification unit 30 notifies the user of a determination result by the image coincidence determination unit 29. The notification unit 30 can notify the user of the determination result, for example, by displaying a message representing the determination result on the monitor 23. By checking the determination result that the current ultrasound image coincides with the past ultrasound image, the user can ascertain that the tomographic plane examined in the past can be reliably examined in the current ultrasound examination. In addition, in a case where the user checks the determination result that the current ultrasound image does not coincide with the past ultrasound image, the user can take measures such as making the body position of the subject coincide with the body position in the past examination, or making a condition such as the posture of the ultrasound probe 1 or the degree of pressing the ultrasound probe 1 against the breast coincide with the condition in the past examination.

Here, the breast of the subject, which is a target of the ultrasound examination in the present invention, is usually deformed easily due to the body position or the body movement of the subject. Therefore, for example, even in a case where the scanning path coincidence determination unit 28 determines that the current scanning path coincides with the past scanning path, the tomographic plane imaged in the past examination and the tomographic plane currently being imaged may be different from each other due to the deformation of the breast. Since the image coincidence determination unit 29 determines whether or not the current ultrasound image coincides with the past ultrasound image acquired by the image acquisition unit 33 in the past ultrasound examination of the subject, and the notification unit 30 notifies the user of the determination result by the image coincidence determination unit 29, the user can reliably examine the tomographic plane observed in the past ultrasound examination.

In addition, for example, as shown in FIG. 8, the notification unit 30 can also display the current ultrasound image U1 and the past ultrasound image U2 acquired by the image acquisition unit 33 side by side on the monitor 23, and highlight the different portions H1 and H2, for example, by displaying the different portions H1 and H2 in different colors or surrounding the different portions H1 and H2 with frame lines. The user can take measures such as changing the body position of the subject, the posture of the ultrasound probe 1, or the degree of pressing the ultrasound probe 1 to correct the difference between the current ultrasound image U1 and the past ultrasound image U2.

In addition, in a case where the image coincidence determination unit 29 determines that the current ultrasound image does not coincide with the past ultrasound image, the notification unit 30 can also notify the user of information representing the body position of the subject corresponding to the past ultrasound image, information representing the posture of the ultrasound probe 1, information representing the degree of pressing of the ultrasound probe 1, and the like. The information representing the body position of the subject corresponding to the past ultrasound image, the information representing the posture of the ultrasound probe 1, the information representing the degree of pressing of the ultrasound probe 1, and the like can be stored in the image memory 24 in advance in association with the past ultrasound image, for example, and the notification unit 30 can read out these pieces of information from the image memory 24. The user can accurately take measures such as changing the body position of the subject, the posture of the ultrasound probe 1, or the degree of pressing of the ultrasound probe 1 by checking the information representing the body position of the subject corresponding to the past ultrasound image, the information representing the posture of the ultrasound probe 1, the information representing the degree of pressing of the ultrasound probe 1, and the like.

The display controller 22 performs predetermined processing on the ultrasound image acquired by the image acquisition unit 33, the notification content by the notification unit 30, and the like under the control of the body controller 31, and displays the ultrasound image, the notification content, and the like on the monitor 23.

The monitor 23 displays the ultrasound image acquired by the image acquisition unit 33, the notification content by the notification unit 30, and the like under the control of the display controller 22, and has, for example, a display device such as a liquid crystal display (LCD) or an organic electroluminescence display (organic EL display).

The input device 32 is an input device used by the user to perform an input operation and is configured by, for example, a device such as a keyboard, a mouse, a trackball, a touchpad, and a touch sensor disposed in a state of being superimposed on the monitor 23.

In the present embodiment, each processing in the processor 34 is executed by any computer. In addition, any computer may execute these processes using a processor as hardware, a program as software, or a combination thereof. In such a case, the processor is configured to execute various types of processing in the present embodiment in cooperation with the program, and may function as each unit or each means in the present embodiment. Further, the execution order of the processes by the processor is not limited to the order described and may be changed as appropriate. Any computer may be a general purpose computer, a special purpose computer, a workstation, or other system capable of executing each process.

The processor 34 may be composed of one or a plurality of pieces of hardware, and types of hardware are not limited. For example, the processor 34 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). Additionally, the type of hardware may be a combination of different types of hardware. In a case where a plurality of pieces of hardware are configured to execute one or a plurality of types of processing of a processor, the plurality of pieces of hardware may be present in apparatuses physically separated from each other, or may be present in the same apparatus. In addition, in any of the embodiments, the order of each processing by the processor 34 is not limited to the above order, and may be appropriately changed. In addition, hardware is implemented in a form of an electric circuit (circuitry) in which circuit elements, such as semiconductor elements, are combined.

Further, the program may be software, such as firmware or a microcode. In addition, the program may be, for example, a program module group, and each function thereof may be realized by a processor configured to execute each function. The program may be a program code or a plurality of code segments stored in one or a plurality of non-transitory computer-readable media (for example, storage media or other storages). The program may be divided and saved in a plurality of non-transitory computer-readable media present in apparatuses physically separated from each other. The program code or the code segment may represent any combination of procedures, functions, subprograms, routines, subroutines, modules, software packages, classes, instructions, data structures, or program statements. The program code or code segments may be connected to other code segments or to hardware circuits by transmitting and receiving information, data, arguments, parameters, or contents of memory.

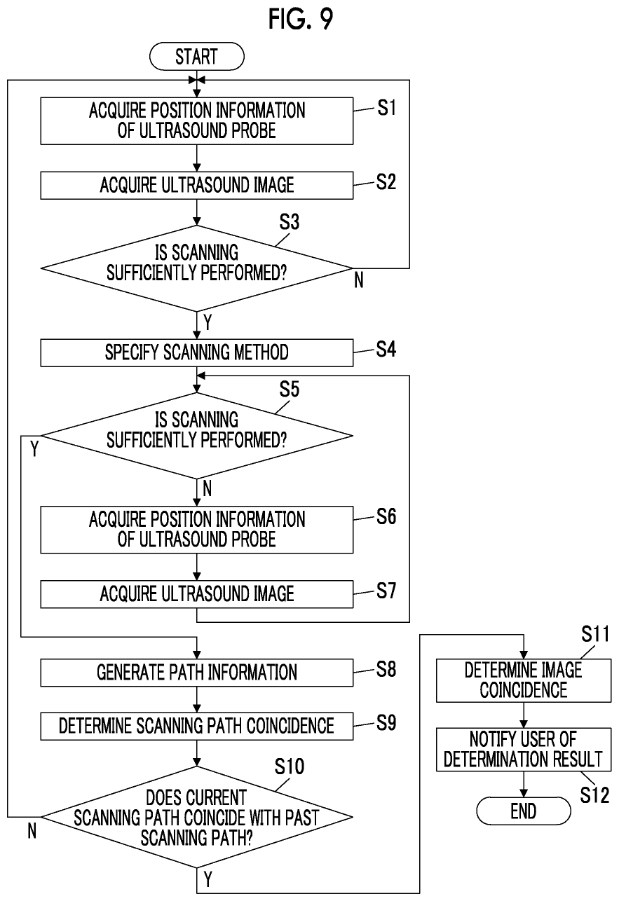

Hereinafter, an operation of the ultrasound diagnostic apparatus according to first embodiment will be described with reference to a flowchart shown in FIG. 9.

In step S1, the user disposes the ultrasound probe 1 on the breast of the subject, and the position sensor 13 acquires the position information of the ultrasound probe 1. The position sensor 13 can acquire the position coordinates and the posture of the ultrasound probe 1 in the three-dimensional space of the ultrasound probe 1, as the position information of the ultrasound probe 1.

In step S2, the image acquisition unit 33 acquires the ultrasound image representing the breast of the subject. In this case, under the control of the body controller 31, the transmission and reception of the ultrasound from the plurality of transducers of the transducer array 11 are started in accordance with the drive signal from the pulser 41 of the transmission and reception circuit 12 of the ultrasound probe 1, the ultrasound echo from the subject is received by the plurality of transducers of the transducer array 11, and the reception signal as the analog signal is output to the amplification unit 42, is amplified, and then is subjected to the AD conversion via the AD conversion unit 43 to acquire the reception data.

The reception focus processing is performed on the reception data by the beam former 44, the sound ray signal generated by the reception focusing processing is transmitted to the image generation unit 21 of the apparatus body 2, and thus the ultrasound image is generated by the image generation unit 21. In this case, the signal processing unit 45 of the image generation unit 21 performs the correction of the attenuation in accordance with the depth of the reflection position of the ultrasound and the envelope detection processing on the sound ray signal, the DSC 46 performs the conversion into the image signal in accordance with the normal television signal scanning method, and the image processing unit 47 performs various types of necessary image processing such as gradation processing.

In step S3, the body controller 31 determines whether or not the scanning is sufficiently performed by the scanning method specifying unit 25 to specify the scanning method of the current ultrasound examination for the breast of the subject, that is, whether or not the ultrasound images of the sufficient number of frames are acquired. The number of frames required for determining that the scanning is sufficiently performed is determined by the method of specifying the scanning method by the scanning method specifying unit 25. For example, in a case in which the scanning method is specified by the input of the user via the input device 32, the number of frames required for the determination is zero. In addition, for example, in a case in which the scanning method is determined based on the trajectory of the ultrasound probe 1 within a predetermined time from the start of the scanning, the number of frames required for the determination is a certain number of 2 or more. The scanning method by the scanning method specifying unit 25 may be set in advance, or may be set by the user via the input device 32.

The processing of step S1 to step S3 is repeated as long as it is determined in step S3 that the scanning is not sufficiently performed. In a case in which it is determined in step S3 that the scanning is sufficiently performed, the processing proceeds to step S4.

In step S4, the scanning method specifying unit 25 specifies the scanning method of the ultrasound probe 1 for the breast. In this case, the scanning method specifying unit 25 can specify the scanning method based on, for example, the information input by the user via the input device 32. The scanning method specifying unit 25 can also specify the scanning method based on at least one of the position information of the ultrasound probe 1 acquired by the position sensor 13 in step S1 or the ultrasound image acquired by the image acquisition unit 33 in step S2.

In step S5, the body controller 31 determines whether or not the scanning is sufficiently performed to generate the path information of the ultrasound probe 1 by the path information generation unit 26, that is, whether or not the ultrasound images of a sufficient number of frames are acquired. The number of frames required to determine that the scanning is sufficiently performed is determined by the content of the path information generated by the path information generation unit 26. The content of the path information generated by the path information generation unit 26 may be set in advance, or may be set by the user via the input device 32.

In a case in which it is determined in step S5 that the scanning is not sufficiently performed, the processing proceeds to step S6. In step S6, the position sensor 13 newly acquires the position information of the ultrasound probe 1 in the same manner as in step S1. In subsequent step S7, the image acquisition unit 33 newly acquires the ultrasound image of the breast of the subject in the same manner as in step S2. The processing of steps S5 to S7 is repeated as long as it is determined in step S5 that the scanning is not sufficiently performed. In a case in which it is determined in step S5 that the scanning is sufficiently performed, the processing proceeds to step S8.

In step S8, the path information generation unit 26 generates the path information related to the scanning path of the ultrasound probe 1 that is used for scanning in accordance with the scanning method specified in step S4, based on the position information of the ultrasound probe 1 acquired in step S1 or step S6. The path information generation unit 26 can generate the path information based on, for example, the position information of the ultrasound probe 1 continuously acquired in step S1 and step S6 in association with the scanning of the ultrasound probe 1. The path information generation unit 26 can also generate the path information based on, for example, the discrete position information of the ultrasound probe 1, such as the position coordinates of the ultrasound probe 1 corresponding to the inflection points of the scanning path, which are acquired in step S1 and step S6.

In step S9, the scanning path coincidence determination unit 28 determines whether or not the current scanning path coincides with the past scanning path in the past ultrasound examination of the subject, based on the path information generated in step S8. In this case, the scanning path coincidence determination unit 28 can determine whether or not the current scanning path coincides with the past scanning path based on the past path information stored in the path information memory 27. More specifically, the scanning path coincidence determination unit 28 can calculate, for example, a difference between the past path information stored in the path information memory 27 and the current scanning path, and determine that the current scanning path coincides with the past scanning path in a case in which the calculated difference is equal to or less than a predetermined difference threshold value.

In step S10, the body controller 31 controls the ultrasound diagnostic apparatus to perform different processing depending on whether or not it is determined in step S9 that the current scanning path coincides with the past scanning path. In a case in which it is determined in step S9 that the current scanning path does not coincide with the past scanning path, the body controller 31 returns the processing to step S1 in step S10. Thereafter, the processing of step S2 to step S9 is performed again. In a case in which it is determined in step S9 that the current scanning path coincides with the past scanning path, the body controller 31 advances the processing to step S11 in step S10.

In step S11, the image coincidence determination unit 29 determines whether or not the current ultrasound image acquired in step S2 and step S7 coincides with the past ultrasound image acquired in the past ultrasound examination of the subject. The image coincidence determination unit 29 can determine the coincidence between the current ultrasound image and the past ultrasound image by, for example, comparing the current ultrasound image and the past ultrasound image acquired in correspondence with at least one location on the scanning path determined to coincide with each other in step S9.

In step S12, the notification unit 30 notifies the user of the determination result in step S11. The notification unit 30 can notify the user of the determination result, for example, by displaying a message representing the determination result on the monitor 23. In addition, for example, as shown in FIG. 8, the notification unit 30 can also display the current ultrasound image U1 acquired in step S2 and step S7 and the past ultrasound image U2 read out from the image memory 24 side by side on the monitor 23 and highlight the locations H1 and H2 that are different from each other, for example, by displaying the locations H1 and H2 in different colors or surrounding the locations H1 and H2 with frame lines.

In a case in which the user is notified that the current ultrasound image does not coincide the past ultrasound image in step S12, the user can take measures such as changing the body position of the subject, the posture of the ultrasound probe 1, or the degree of pressing of the ultrasound probe 1 to correct the difference between the current ultrasound image U1 and the past ultrasound image U2.

In a case in which the processing of step S12 is completed in this manner, the operation of the ultrasound diagnostic apparatus according to the flowchart of FIG. 9 is completed.

As described above, with the ultrasound diagnostic apparatus according to the first embodiment of the present invention, the position sensor 13 acquires the position information of the ultrasound probe 1, the scanning method specifying unit 25 specifies the scanning method of the ultrasound probe 1 for the breast, the path information generation unit 26 generates the path information related to the scanning path of the ultrasound probe 1 that is used for scanning in accordance with the scanning method specified by the scanning method specifying unit 25 based on the position information acquired by the position sensor 13, the scanning path coincidence determination unit 28 determines whether or not the current scanning path coincides with the past scanning path based on the path information generated by the path information generation unit 26, the image coincidence determination unit 29 determines whether or not the current ultrasound image coincides with the past ultrasound image, and the notification unit 30 notifies the user of the determination result by the image coincidence determination unit 29. Therefore, the user can reliably examine the tomographic plane observed in the past ultrasound examination.

A case has been described in which the transmission and reception circuit 12 is provided in the ultrasound probe 1, but the transmission and reception circuit 12 may be provided in the apparatus body 2.

Further, a case has been described in which the image generation unit 21 is provided in the apparatus body 2, but the image generation unit 21 may be provided in the ultrasound probe 1.

The apparatus body 2 may be a so-called stationary type, a portable type that is easy to carry, or a so-called handheld type that is configured by, for example, a smartphone or a tablet type computer. As described above, the type of the device that constitutes the apparatus body 2 is not particularly limited.

It should be noted that, although the description has been made in which the apparatus body 2 comprises the image memory 24 and the path information memory 27, the image memory 24 and the path information memory 27 may be provided in an external server device (not shown).

It should be noted that, although the description has been made in which the image acquisition unit 33 acquires the B-mode image as the ultrasound image, the image acquisition unit 33 can further acquire a so-called Doppler image or an elastic image. Here, the elastic image refers to an image acquired by so-called elastography. In such a case, the image coincidence determination unit 29 can use the Doppler image or the elastic image as the ultrasound image used for determining the coincidence.

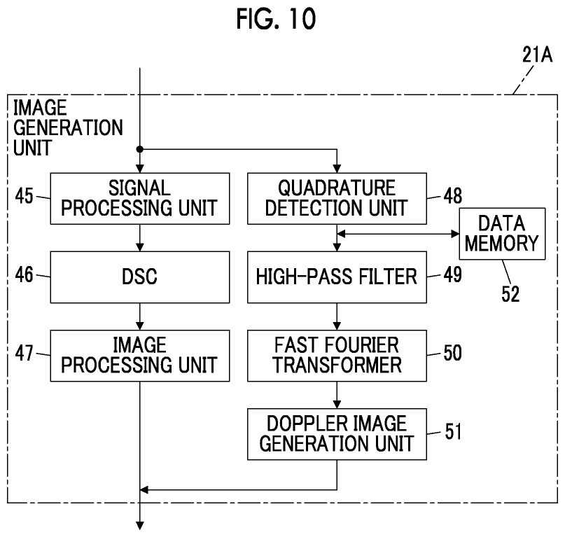

In a case in which the Doppler image is generated as the ultrasound image, the apparatus body 2 can comprise, for example, an image generation unit 21A shown in FIG. 10. The image generation unit 21A further comprises a quadrature detection unit 48, a high pass filter 49, a fast Fourier transform unit 50, a Doppler image generation unit 51, and a data memory 52 in the image generation unit 21 shown in FIG. 3. The quadrature detection unit 48 is connected to the transmission and reception circuit 12 in parallel with the signal processing unit 45. In addition, a high-pass filter 49, a fast Fourier transform unit 50, and a Doppler image generation unit 51 are sequentially connected to the quadrature detection unit 48. The Doppler image generation unit 51 is connected to the display controller 22, the image memory 24, the scanning method specifying unit 25, and the image coincidence determination unit 29, similarly to the image processing unit 47. In addition, the data memory 52 is connected to the quadrature detection unit 48.

The quadrature detection unit 48 mixes the acoustic signal received from the transmission and reception circuit 12 with a carrier signal of a reference frequency to orthogonally detect the acoustic signal and convert the acoustic signal into a complex signal.

The high-pass filter 49 functions as a so-called wall filter, and removes a frequency component derived from the motion of the body tissue inside the subject, from the complex data generated by the quadrature detection unit 48.

The fast Fourier transformer 50 performs a Fourier transform on the complex data of a plurality of sample points to perform frequency analysis, obtains the blood flow velocity, and generates a spectrum signal.

The Doppler image generation unit 51 generates a Doppler image by aligning the spectrum signals generated by the fast Fourier transform unit 50 on a time axis and representing the magnitude of each frequency component as brightness. In the Doppler image, a lateral axis represents the time axis, a vertical axis represents the Doppler shift frequency, that is, the flow velocity, and the brightness of the waveform represents the power in each frequency component.

The data memory 52 stores the complex signal converted from the reception data by the quadrature detection unit 48. For example, recording media such as a flash memory, an HDD, an SSD, an FD, an MO disc, an MT, a RAM, a CD, a DVD, an SD card, and a USB memory can be used as the data memory 52.

The processing of generating the Doppler image by the quadrature detection unit 48, the high pass filter 49, the fast Fourier transform unit 50, and the Doppler image generation unit 51 is performed by transmitting a drive signal to the transducer array 11 to transmit the ultrasound waves in a pulsed manner under the control of the transmission/reception circuit 12 and the body controller 31, and the Doppler image is generated in a so-called Doppler gate set on the B-mode image by the input of the user via the input device 32 or the like.

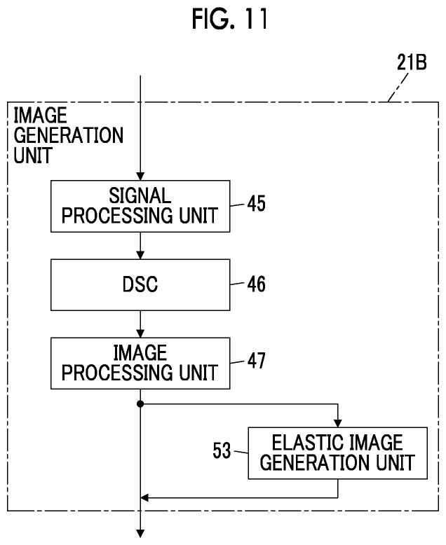

In a case in which the elastic image is generated as the ultrasound image, the apparatus body 2 can comprise, for example, an image generation unit 21B shown in FIG. 11. The image generation unit 21B further comprises an elastic image generation unit 53 in the image generation unit 21 shown in FIG. 3. The elastic image generation unit 53 is connected to the image processing unit 47. The elastic image generation unit 53 is connected to the display controller 22, the image memory 24, the scanning method specifying unit 25, and the image coincidence determination unit 29, similarly to the image processing unit 47.

The elastic image generation unit 53 generates an elastic image representing a distribution of hardness of the tissue in the subject by a so-called elastography method. The elastic image generation unit 53 can generate the elastic image by, for example, calculating a distribution of distortion of the tissue shown in the B-mode image based on a difference between the B-mode image before the ultrasound probe 1 is pressed against the body surface of the subject and the B-mode image after the ultrasound probe 1 is pressed against the body surface of the subject, and converting the calculated distribution of distortion into a distribution of hardness using distortion of a reference object having a known elastic modulus, such as an elastic coupler mounted on the distal end of the ultrasound probe 1. In addition, the elastic image generation unit 53 can also generate the elastic image by, for example, irradiating a plurality of locations in the subject with the ultrasound pulses from the ultrasound probe 1 to generate a shear wave, and measuring the distribution of the hardness of the tissue based on a propagation velocity of the shear wave in the tissue in the subject. The generated elastic image is superimposed on the B-mode image in a state in which a color is assigned to each pixel according to the hardness of the tissue, and is displayed on the monitor 23, for example.

Although the position sensor 13 that is attached to the ultrasound probe 1 or that is installed independently of the ultrasound probe 1 has been described as the probe position information acquisition unit that acquires the position information of the ultrasound probe 1, for example, the apparatus body 2 can also comprise a probe position information acquisition unit that acquires the position information of the ultrasound probe 1 based on the ultrasound image, as a part of the processor 34. In this case, the probe position information acquisition unit can acquire the position information of the ultrasound probe 1 by, for example, inputting the ultrasound image acquired by the image acquisition unit 33 to a trained model in machine learning that has learned in advance a relationship between the image pattern of the anatomical structure in the subject and the imaging position and the posture of the ultrasound probe 1.

In addition, in a case in which the reference figure used as a so-called augmented reality (AR) marker is installed in the housing of the ultrasound probe 1, the ultrasound diagnostic apparatus can comprise a probe position information acquisition unit that is composed of an optical camera that captures an optical image of the ultrasound probe 1 and a reference figure detection unit that is a part of the processor 34 and that acquires the position information of the ultrasound probe 1 by detecting the reference figure shown in the optical image.

Although an example in which the notification unit 30 notifies the user of the display on the monitor 23 has been described, the method of notifying the user is not particularly limited thereto. For example, in a case in which the ultrasound diagnostic apparatus comprises a speaker (not shown), the notification unit 30 can notify the user of the display via the speaker.

### Second Embodiment

In general, the breast of the subject may be deformed by being pressed by the ultrasound probe 1. As a result, the scanning path of the ultrasound probe 1 may also be deformed. The ultrasound diagnostic apparatus can correct the path information related to the scanning path of the ultrasound probe 1 that has changed in accordance with the deformation of the breast.

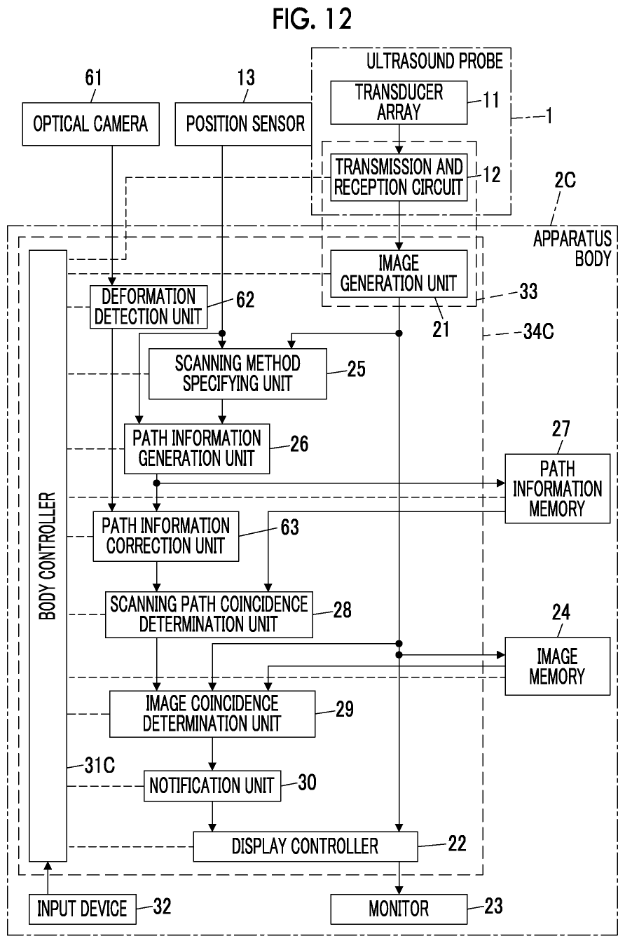

FIG. 12 shows a configuration of an ultrasound diagnostic apparatus according to a second embodiment of the present invention. The ultrasound diagnostic apparatus of the second embodiment further comprises an optical camera 61 and comprises an apparatus body 2C instead of the apparatus body 2, with respect to the ultrasound diagnostic apparatus of the first embodiment shown in FIG. 1. The apparatus body 2C further comprises a deformation detection unit 62 and a path information correction unit 63, and comprises a body controller 31C instead of the body controller 31, as compared with the apparatus body 2 according to first embodiment.

The deformation detection unit 62 is connected to the optical camera 61. The path information correction unit 63 is connected to the path information generation unit 26 and the deformation detection unit 62. The path information correction unit 63 is connected to the scanning path coincidence determination unit 28. In addition, the deformation detection unit 62 and the path information correction unit 63 are connected to the body controller 31C. In addition, a processor 34C for the apparatus body 2C is configured by the image generation unit 21, the display controller 22, the scanning method specifying unit 25, the path information generation unit 26, the scanning path coincidence determination unit 28, the image coincidence determination unit 29, the notification unit 30, the body controller 31C, the deformation detection unit 62, and the path information correction unit 63.

The optical camera 61 includes, for example, an image sensor, such as a so-called charge coupled device (CCD) image sensor or a so-called complementary metal-oxide-semiconductor (CMOS) image sensor, and images the body surface of the subject and the ultrasound probe 1 disposed on the body surface of the subject to acquire an optical image. The optical camera 61 sends the acquired optical image to the deformation detection unit 62.

The deformation detection unit 62 detects the deformation of the breast of the subject caused by the pressing of the ultrasound probe 1 based on the optical image acquired by the optical camera 61. The deformation detection unit 62 can detect the deformation of the breast, for example, by detecting the breast of the subject shown in the optical image and comparing the shape of the breast before the deformation with the shape of the deformed breast during the ultrasound examination. In addition, the deformation detection unit 62 can also detect the deformation of the breast by, for example, detecting the body position of the subject shown in the optical image and determining that the breast is deformed by gravity in a case where the detected body position of the subject is different from, for example, a reference body position such as a so-called supine posture. The deformation detection unit 62 can detect the breast of the subject or the body position of the subject from the optical image by, for example, a method using a so-called template coincidence or a trained model in machine learning.

The path information correction unit 63 corrects the current path information generated by the path information generation unit 26 based on the deformation of the breast detected by the deformation detection unit 62. The path information correction unit 63 can correct the current path information by, for example, representing the scanning path of the ultrasound probe 1 in the path information generated by the path information generation unit 26 by a Bézier curve, setting the position of the characteristic structure such as the nipple or mole of the breast of the subject shown in the optical image as a so-called anchor point of the Bézier curve, and deforming the Bézier curve representing the scanning path in accordance with the displacement of the anchor point due to the deformation of the breast.

In addition, the path information correction unit 63 can generate a three-dimensional model of the breast of the subject before the deformation based on, for example, the optical images of the plurality of frames, and can dispose the scanning path in the current path information generated by the path information generation unit 26 on the surface of the three-dimensional model. The path information correction unit 63 can also correct the current path information by deforming the scanning path together with the three-dimensional model in accordance with the deformation of the breast of the subject shown in the optical image.

The scanning path coincidence determination unit 28 determines the coincidence of the scanning paths based on the current path information corrected by the path information correction unit 63 and the past path information. As a result, the scanning path coincidence determination unit 28 can more accurately determine the coincidence of the scanning paths based on the current path information and the past path information in consideration of the deformation of the breast of the subject.

As described above, with the ultrasound diagnostic apparatus according to the second embodiment, the deformation detection unit 62 detects the deformation of the breast of the subject, and the path information correction unit 63 corrects the current path information generated by the path information generation unit 26 based on the deformation of the breast detected by the deformation detection unit 62. Therefore, the coincidence of the scanning paths can be more accurately determined based on the current path information and the past path information, and the tomographic plane observed in the past ultrasound examination can be more reliably examined.

It should be noted that, although the description has been made in which the deformation detection unit 62 detects the deformation of the breast of the subject based on the optical image more acquired by the optical camera 61, the method in which the deformation detection unit 62 detects the breast of the subject is not particularly limited thereto. The deformation detection unit 62 can detect the deformation of the breast of the subject by, for example, detecting the tissue in the subject from the ultrasound images of the plurality of frames acquired by the image acquisition unit 33 and calculating the displacement of the detected tissue in the subject. In addition, in a case in which a pressure sensor (not shown) for measuring the pressure applied to the breast of the subject by the ultrasound probe 1 is attached to the distal end of the ultrasound probe 1, the deformation detection unit 62 can also detect the deformation of the breast of the subject, for example, by determining whether or not the pressure measured by the pressure sensor exceeds a predetermined pressure threshold value.

### Third Embodiment

During the ultrasound examination of the breast of the subject, the scanning path of the ultrasound probe 1 may deviate from the scanning path corresponding to the scanning method specified by the scanning method specifying unit 25 due to some reason. In this case, the ultrasound diagnostic apparatus can propose the corrected path information to the user.

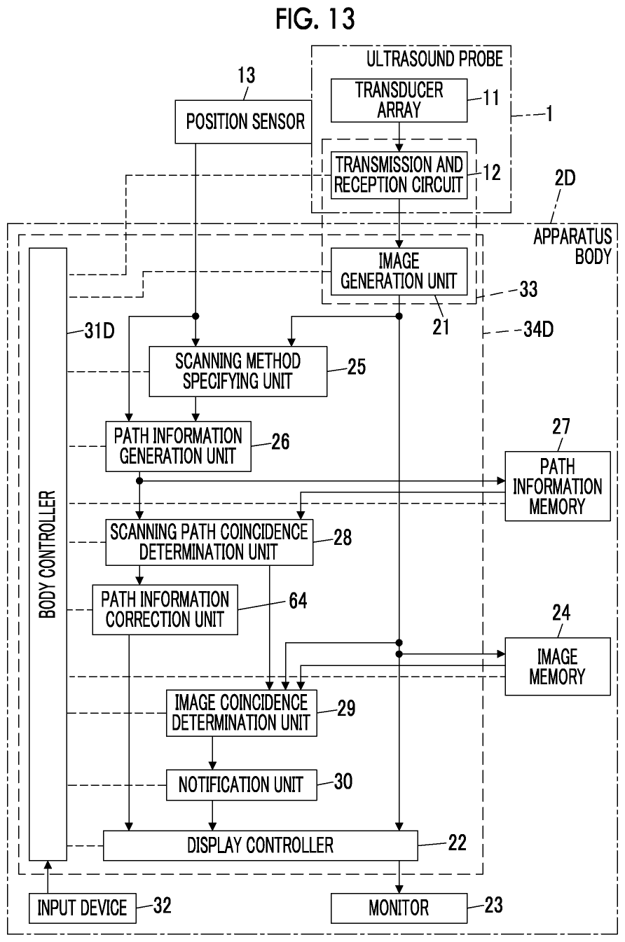

FIG. 13 illustrates a configuration of an ultrasound diagnostic apparatus of a third embodiment. The ultrasound diagnostic apparatus according to the third embodiment comprises an apparatus body 2D instead of the apparatus body 2 of the ultrasound diagnostic apparatus according to the first embodiment shown in FIG. 1. The apparatus body 2D further comprises a path information correction unit 64 and comprises a body controller 31D instead of the body controller 31, in the apparatus body 2 according to first embodiment. The path information correction unit 64 is connected to the scanning path coincidence determination unit 28. The path information correction unit 64 is connected to the display controller 22 and the body controller 31D. In addition, a processor 34D for the apparatus body 2D is configured by the image generation unit 21, the display controller 22, the scanning method specifying unit 25, the path information generation unit 26, the scanning path coincidence determination unit 28, the image coincidence determination unit 29, the notification unit 30, the body controller 31D, and the path information correction unit 64.

In a case where the current scanning path deviates from the past scanning path by a predetermined threshold value or more, the path information correction unit 64 corrects the path information generated by the path information generation unit 26 based on the past scanning path, and proposes the corrected path information to the user by, for example, display on the monitor 23. For example, the path information correction unit 64 can calculate a difference between the current scanning path and the past scanning path, and in a case where the calculated difference is equal to or greater than a predetermined threshold value, correct the path information such that the calculated difference is reduced, and propose the corrected path information to the user. The user can cause the ultrasound probe 1 to perform scanning along the scanning path corresponding to the scanning method specified by the scanning method specifying unit 25 while checking the proposed path information.

In a case of proposing the corrected path information, the path information correction unit 64 can present, for example, the movement direction and the inclination direction of the ultrasound probe 1 to the user such that the difference between the current scanning path and the past scanning path is reduced. In addition, the path information correction unit 64 can also present, for example, an instruction to the user to return the position of the ultrasound probe 1 to a position where the scanning path of the ultrasound probe 1 starts to deviate from the scanning path corresponding to the scanning method specified by the scanning method specifying unit 25, or to the start position of the scanning path corresponding to the scanning method specified by the scanning method specifying unit 25, and to perform scanning again.

As described above, with the ultrasound diagnostic apparatus according to the third embodiment of the present invention, in a case where the current scanning path deviates from the past scanning path by a predetermined threshold value or more, the path information correction unit 64 corrects the path information generated by the path information generation unit 26 based on the past scanning path, and proposes the corrected path information to the user. Therefore, the user can cause the ultrasound probe 1 to perform scanning accurately along the scanning path corresponding to the scanning method specified by the scanning method specifying unit 25.

It should be noted that, although it has been described that the apparatus body 2D in the third embodiment is configured by adding the path information correction unit 64 to the apparatus body 2 in the first embodiment, the path information correction unit 64 can also be added to the apparatus body 2C in the second embodiment.

In addition, although it has been described that the path information correction unit 64 proposes the path information to the user by display on the monitor 23, the method of proposing the path information to the user by the path information correction unit 64 is not particularly limited thereto. In a case where the ultrasound diagnostic apparatus comprises a speaker (not shown), the path information correction unit 64 can propose, for example, the movement direction, the inclination direction, and the like of the ultrasound probe 1 to the user via the speaker.

### EXPLANATION OF REFERENCES

1: ultrasound probe
2, 2C, 2D: apparatus body
11: transducer array
12: transmission and reception circuit
13: position sensor
21, 21A, 21B: image generation unit
22: display controller
23: monitor
24: image memory
25: scanning method specifying unit
26: path information generation unit
27: path information memory
28: scanning path coincidence determination unit
29: image coincidence determination unit
30: notification unit
31, 31C, 31D: body controller
32: input device
33: image acquisition unit
34, 34C, 34D: processor
41: pulser
42: amplification unit
43: AD conversion unit
44: beam former
45: signal processing unit
46: DSC
47: image processing unit
48: quadrature detection unit
49: high-pass filter
50: fast Fourier transformer
51: Doppler image generation unit
52: data memory
53: elastic image generation unit
61: optical camera
62: deformation detection unit
63: path information correction unit
64: path information correction unit
BM: body mark
H1, H2: portions that are different from each other
P1, P2, P3, P4: path
U1, U2: ultrasound image

What is claimed is:

1. An ultrasound diagnostic apparatus that performs an ultrasound examination of a breast of a subject by scanning the breast with an ultrasound probe, the ultrasound diagnostic apparatus comprising:
a processor configured to:
acquire an ultrasound image by transmitting and receiving an ultrasound beam using the ultrasound probe;
acquire position information of the ultrasound probe;
specify a scanning method of the ultrasound probe with respect to the breast;
generate, based on the position information, path information related to a scanning path of the ultrasound probe used for scanning in accordance with the scanning method;
determine, based on the path information, whether or not a current scanning path coincides with a past scanning path in a past ultrasound examination of the subject;
upon determining that the current scanning path coincides with the past scanning path, determine whether or not a current ultrasound image coincides with a past ultrasound image in the past ultrasound examination of the subject; and
notify a user of a determination result of whether the current ultrasound image coincides with the past ultrasound image.

2. The ultrasound diagnostic apparatus according to claim 1, further comprising:
an image memory configured to store the ultrasound image; and
a path information memory configured to store the path information,
wherein the processor is configured to::
determine whether or not the current scanning path coincides with the past scanning path based on the path information stored in the path information memory; and
determine whether or not the current ultrasound image coincides with the past ultrasound image based on the ultrasound image stored in the image memory.

3. The ultrasound diagnostic apparatus according to claim 1,
wherein the processor is configured to specify the scanning method based on information input by the user.

4. The ultrasound diagnostic apparatus according to claim 2,
wherein the processor is configured to specify the scanning method based on information input by the user.

5. The ultrasound diagnostic apparatus according to claim 1,
wherein the processor is configured to specify the scanning method based on at least one of the ultrasound image or the position information.

6. The ultrasound diagnostic apparatus according to claim 2,
wherein the processor is configured to specify the scanning method based on at least one of the ultrasound image or the position information.

7. The ultrasound diagnostic apparatus according to claim 1,
wherein the processor is configured to generate the path information based on the position information continuously acquired in association with scanning using the ultrasound probe.

8. The ultrasound diagnostic apparatus according to claim 2,
wherein the processor is configured to generate the path information based on the position information continuously acquired in association with scanning using the ultrasound probe.

9. The ultrasound diagnostic apparatus according to claim 3,
wherein the processor is configured to generate the path information based on the position information continuously acquired in association with scanning using the ultrasound probe.

10. The ultrasound diagnostic apparatus according to claim 5,
wherein the processor is configured to generate the path information based on the position information continuously acquired in association with scanning using the ultrasound probe.

11. The ultrasound diagnostic apparatus according to claim 1,
wherein the processor is configured to generate the path information based on discrete position information.

12. The ultrasound diagnostic apparatus according to claim 2,
wherein the processor is configured to generate the path information based on discrete position information.

13. The ultrasound diagnostic apparatus according to claim 3,
wherein the processor is configured to generate the path information based on discrete position information.

14. The ultrasound diagnostic apparatus according to claim 5,
wherein the processor is configured to generate the path information based on discrete position information.

15. The ultrasound diagnostic apparatus according to claim 1,
wherein the processor is configured to compare the current ultrasound image with the past ultrasound image both corresponding to at least one location on scanning paths determined to coincide each other, to determine coincidence between the current ultrasound image and the past ultrasound image.

16. The ultrasound diagnostic apparatus according to claim 1,
wherein the processor is configured to:
detect deformation of the breast of the subject;
correct current path information based on the deformation of the breast; and
determine coincidence of the scanning path based on the current path information and past path information.

17. The ultrasound diagnostic apparatus according to claim 1, further comprising:
a monitor,
wherein the processor is configured to display, side by side on the monitor, the current ultrasound image and the past ultrasound image; and
highlight portions between the current ultrasound image and the past ultrasound image that differ from each other.

18. The ultrasound diagnostic apparatus according to claim 1,
wherein the processor is configured to, upon determining that the current scanning path deviates from the past scanning path by a predetermined threshold value or more,
correct the path information based on the past scanning path; and
propose the corrected path information to the user.

19. The ultrasound diagnostic apparatus according to claim 1,
wherein the ultrasound image is any of a B-mode image, a Doppler image, or an elasticity image.

20. A control method for an ultrasound diagnostic apparatus that performs an ultrasound examination of a breast of a subject by scanning the breast with an ultrasound probe, the control method comprising:
acquiring an ultrasound image by transmitting and receiving an ultrasound beam using the ultrasound probe;
acquiring position information of the ultrasound probe;
specifying a scanning method of the ultrasound probe with respect to the breast;
generating, based on the acquired position information, path information related to a scanning path of the ultrasound probe used for scanning in accordance with the specified scanning method;
determining, based on the generated path information, whether or not a current scanning path coincides with a past scanning path in a past ultrasound examination of the subject;
in a case where it is determined that the current scanning path coincides with the past scanning path, determining whether or not an acquired current ultrasound image coincides with a past ultrasound image acquired in the past ultrasound examination of the subject; and
notifying a user of a determination result of coincidence between the current ultrasound image and the past ultrasound image.
